(19) United States
(12) Patent Application Publication
(10) Pub. No.: US 2023/0295290 A1
(43) Pub. Date: Sep. 21, 2023

(54) Treatment of Respiratory Disease

(71) Applicant: Eli Lilly and Company, Indianapolis, IN (US)

(72) Inventors: Gaia GALLO, Carmel, IN (US); Venkatesh KRISHNAN, Fishers, IN (US); Sreekumar G. PILLAI, Carmel, IN (US); Brian Jeffrey NICKOLOFF, Carmel, IN (US)

(21) Appl. No.: 17/935,409

(22) Filed: Sep. 26, 2022

Related U.S. Application Data
(63) Continuation of application No. PCT/US2021/026840, filed on Apr. 12, 2021.
(60) Provisional application No. 63/011,430, filed on Apr. 17, 2020.
(60) Provisional application No. 63/063,493, filed on Aug. 10, 2020.

(51) Int. Cl. C07K 16/24; A61P 31/14; A61P 11/00

(52) U.S. Cl. CPC C07K 16/244; A61P 31/14; A61P 11/00; C07K 2317/24; A61K 2039/545

(57) ABSTRACT

Methods of preventing and/or treating ARDS with anti-IL17 antibodies, including dose regimens and pharmaceutical formulations of anti-IL17 antibodies for use in the prevention and/or treatment of ARDS.

### REFERENCE TO AN ELECTRONIC SEQUENCE LISTING

The instant application contains a Sequence Listing which has been submitted electronically in ASCII format and is hereby incorporated by reference in its entirety. Said ASCII copy, created on Sep. 26, 2022, is named X22768USsequencelisting and is 8,156 bytes in size.

The present disclosure relates to the field of medicine. More particularly, the present disclosure relates to methods of preventing and/or treating acute respiratory distress syndrome (ARDS) with an anti-IL17 antibody. The present disclosure also provides dose regimens and pharmaceutical formulations use for the prevention and/or treatment of ARDS.

ARDS is a life-threatening respiratory disease characterized by inflammation of the lungs, which can be widespread and rapid in onset. ARDS has a mortality rate reported as high as 30-40%. Symptoms associated with ARDS include shortness of breath, rapid breathing and bluish skin coloration, in association with disease or injury. Formal diagnosis of ARDS is challenging as scientific and medical definitions have evolved. One definition, known as the “Berlin definition”, relies on radiological imagining of lung and PaO2/FiO2 ratios (Ranieri et al., (June 2012), “Acute respiratory distress syndrome: The Berlin Definition. ARDS Definition Task Force”, JAMA, 307 (23): 2526-33). According to the Berlin definition, ARDS is characterized according to the following factors: acute onset respiratory symptoms following lung insult; unexplained bilateral opacities on chest imaging; respiratory failure (not explained by heart failure or volume overload); decreased PaO2/FiO2 ratio. The Berlin definition also allows for staging of ARDS according to: mild ARDS: 201-300 mmHg (≤39.9 kPa); moderate ARDS: 101-200 mmHg (≤26.6 kPa); and severe ARDS: ≤100 mmHg (≤13.3 kPa). However, even in those who recover, although lung function may gradually improve over a period of six months to a year, patients may be left with significant scarring and lower than normal lung volumes.

There are presently no approved treatments for preventing and/or treating ARDS. A complicating factor in developing a therapy for ARDS is that ARDS and associated mortalities develop from a number of diseases and injuries, including interstitial lung disease, viral insult such as coronavirus disease 2019 (COVID-19), caused by SARS-CoV-2 virus, and middle eastern respiratory syndrome (MERS), and therapy induced insult such as CAR-T therapy induced ARDS. Additionally, ARDS involves a number of molecular pathways involving numerous immune and epithelial targets. Further, complex dysregulation of the body's own immune response, following disease or injury (for example, as in COVID-19), leading to a phenomenon known as cytokine storm has also been associated with ARDS. As such, there remains an urgent and unmet need for a prevention and/or treatment of ARDS. As with all therapeutic treatments, safety and toxicity remain a limitation, thus any improved treatments must not be attendant on unacceptable safety and toxicity profiles. The present disclosure provides a method of preventing and/or treating ARDS. More particularly, the present disclosure provides an anti-IL17 antibody, and methods of using the same, for the prevention of and/or treatment of ARDS.

Accordingly, the present disclosure provides a method for the prevention and/or treatment for ARDS which addresses one or more of the challenges described herein. In some embodiments, the methods of preventing and/or treating ARDS is provided comprising administering to a patient in need of such treatment, a therapeutically effective amount of an anti-IL17 antibody, or a pharmaceutical formulation thereof. In some embodiments, the anti-IL17 antibody specifically binds human IL17A. In more specific embodiments, the anti-IL17 antibody comprises a light chain variable region (LCVR) and a heavy chain variable region (HCVR), wherein said LCVR is the amino acid sequence of SEQ ID NO: 2 and said HCVR is the amino acid sequence of SEQ ID NO: 3. In even more specific embodiments, the anti-IL17 antibody comprises a light chain (LC) and a heavy chain (HC), wherein said LC is the amino acid sequence of SEQ ID NO: 4 and said HC is the amino acid sequence of SEQ ID NO: 5. In even more specific embodiments, the anti-IL17 antibody is ixekizumab.

According to a more particular embodiment, a method of preventing ARDS in a patient is provided, comprising administering to said patient a therapeutically effective amount of an anti-IL17 antibody, or a pharmaceutical formulation thereof. In some embodiments, the patient is at risk of developing ARDS. In some embodiments, the patient has a respiratory insult. In some embodiments, the respiratory insult is a respiratory disease. According to some embodiments, the respiratory insult is a respiratory injury.

According to additional embodiments, a method of treating ARDS in a patient is provided comprising administering to said patient a therapeutically effective amount of an anti-IL17 antibody, or a pharmaceutical formulation thereof. In some embodiments, the patient is diagnosed as having mild ARDS. In some embodiments, the patient is diagnosed as having moderate ARDS. In some embodiments, the patient is diagnosed as having severe ARDS. In some embodiments, the patient is diagnosed as having one of mild, moderate or severe ARDS according to the Berlin definition.

According to some embodiments of the methods provided herein, the patient has a viral infection. In some embodiments, the viral infection is a coronavirus infection. In even more particular embodiments the coronavirus is SARS-CoV-2. According to some embodiments of the methods provided herein, the patient has pneumonia. According to some embodiments of the methods provided herein, the patient has asthma. According to some embodiments the patient has chronic obstructive pulmonary disease (COPD). According to some embodiments the patient has pulmonary fibrosis. According to some embodiments the patient has interstitial lung disease.

According to embodiments provided herein, the anti-IL17 antibody specifically binds human IL17A. In more particular embodiments, the anti-IL17 antibody comprises a light chain variable region (LCVR) and a heavy chain variable region (HCVR), wherein said LCVR is the amino acid sequence of SEQ ID NO: 2 and said HCVR is the amino acid sequence of SEQ ID NO: 3. In even more particular embodiments, the anti-IL17 antibody comprises a light chain (LC) and a heavy chain (HC), wherein said LC is the amino acid sequence of SEQ ID NO: 4 and said HC is the amino acid sequence of SEQ ID NO: 5. In even more particular embodiments, the anti-IL17 antibody is ixekizumab.

According to embodiments of the methods provided herein, administering comprises administering a pharmaceutical formulation of the anti-IL17 antibody, said pharmaceutical formulation comprising the anti-IL17 antibody at a concentration of about 80 mg/mL. In other embodiments, administering comprises administering a pharmaceutical formulation of the anti-IL17 antibody, said pharmaceutical formulation comprising the anti-IL17 antibody at a concentration of about 160 mg/ml. According to some embodiments, the pharmaceutical formulation further comprises a citrate buffer at a concentration of about 20 mM, sodium chloride at a concentration of about 200 mM, polysorbate-80 at a concentration of about 0.03% (w/v) and pH at about 5.7. In other embodiments, the pharmaceutical formulation further comprises sucrose at a concentration of about 234 mM, polysorbate-80 at a concentration of about 0.03% (w/v) and pH at about 5.7, wherein the pharmaceutical formulation is substantially free of an ionic tonicity excipient and substantially free of L-amino acid excipients.

According to some embodiments of the methods provided herein, administering comprises administering a dose of about 160 mg of the anti-IL17 antibody. In some embodiments, administering comprises administering a dose of about 80 mg of the anti-IL17 antibody. In some embodiments, administering further comprises administering about 160 mg of the anti-IL17 antibody at each of weeks 2, 3 and 4 following the initial dose (e.g., 160Q1). In some embodiments, administering further comprises administering about 80 mg of the anti-IL17 antibody at each of weeks 1, 2, 3 and 4 following the initial dose (e.g., 80Q1). In other embodiments, administering further comprises administering about 160 mg of the anti-IL17 antibody or pharmaceutical formulation thereof at each of Weeks 2 and 4 following the initial dose (e.g., 160Q2). In other embodiments, administering further comprises administering about 80 mg of the anti-IL17 antibody or pharmaceutical formulation thereof at each of Weeks 2 and 4 following the initial dose (e.g., 80Q2). In yet other embodiments, administering further comprises administering about 160 mg of the anti-IL17 antibody or pharmaceutical formulation thereof at week 4 following the initial dose (e.g., 160Q4). In even further embodiments, administering further comprises administering about 80 mg of the anti-IL17 antibody or pharmaceutical formulation thereof at week 4 following the initial dose (e.g., 80Q4). In some embodiments herein, the step of administering comprises subcutaneous administration.

Additionally, an anti-IL17 antibody, or a pharmaceutical formulation thereof, for use in the manufacture of a medicament for the prevention of ARDS is provided herein. Other embodiments provided herein include an anti-IL17 antibody, or a pharmaceutical formulation thereof, for use in the manufacture of a medicament for the treatment of ARDS.

According to some embodiments, provided herein is an anti-IL17 antibody, or a pharmaceutical formulation thereof, for use in the prevention of ARDS, wherein a therapeutically effective amount of the anti-IL17 antibody or a pharmaceutical formulation thereof is administered to a patient in at risk of ARDS. In some embodiments, the patient is at risk of developing ARDS. In some embodiments, the patient has a respiratory insult. In some embodiments, the respiratory insult is a respiratory disease. In some embodiments, the respiratory insult is a respiratory injury.

In some embodiments, provided herein is an anti-IL17 antibody, or a pharmaceutical formulation thereof, for use in the treatment of ARDS, wherein a therapeutically effective amount of the anti-IL17 antibody or a pharmaceutical formulation thereof is administered to a patient in need of treatment for ARDS. In some embodiments, the patient is diagnosed as having mild ARDS. In some embodiments, the patient is diagnosed as having moderate ARDS. In some embodiments, the patient is diagnosed as having severe ARDS. In some embodiments, the patient is diagnosed as having one of mild, moderate or severe ARDS according to the Berlin definition.

According to embodiments of the anti-IL17 antibody, or a pharmaceutical formulation thereof, provided herein, the patient has a viral infection. In some embodiments, the viral infection is a coronavirus infection. In even more particular embodiments the coronavirus is SARS-CoV-2. According to some embodiments provided herein, the patient has pneumonia. According to some embodiments, the patient has asthma. According to some embodiments the patient has COPD. According to some embodiments the patient has pulmonary fibrosis.

According to some embodiments of the anti-IL17 antibody, or a pharmaceutical formulation thereof, the anti-IL17 antibody specifically binds human IL17A. In some more particular embodiments, the anti-IL17 antibody comprises a light chain variable region (LCVR) and a heavy chain variable region (HCVR), wherein said LCVR is the amino acid sequence of SEQ ID NO: 2 and said HCVR is the amino acid sequence of SEQ ID NO: 3. In some embodiments, the anti-IL17 antibody comprises a light chain (LC) and a heavy chain (HC), wherein said LC is the amino acid sequence of SEQ ID NO: 4 and said HC is the amino acid sequence of SEQ ID NO: 5. In even more particular embodiments, the anti-IL17 antibody is ixekizumab. According to some such embodiments, the anti-IL17 antibody, or a pharmaceutical formulation thereof comprise a pharmaceutical formulation of the anti-IL17 antibody, said pharmaceutical formulation comprising the anti-IL17 antibody at a concentration of about 80 mg/mL, citrate buffer at a concentration of about 20 mM, sodium chloride at a concentration of about 200 mM, polysorbate-80 at a concentration in the range of about 0.03% (w/v), and pH at about 5.7. According to some other embodiments, the anti-IL17 antibody, or a pharmaceutical formulation thereof comprise a pharmaceutical formulation of the anti-IL17 antibody, said pharmaceutical formulation comprising the anti-IL17 antibody at a concentration of about 80 mg/mL, sucrose at a concentration of about 234 mM, polysorbate-80 at a concentration of about 0.03% (w/v) and pH at about 5.7, wherein the pharmaceutical formulation is substantially free of an ionic tonicity excipient and substantially free of L-amino acid excipients.

As referred to herein, the terms “individual,” “subject,” and “patient,” used interchangeably herein, refer to a human. In a certain embodiment, the patient, is further characterized with a respiratory disease, for example, caused by an injury, insult such as SARS-CoV-2 viral infection or disease such as COVID-19 disease that would benefit from a decreased bioactivity of IL-17. In some embodiments, the patient is further characterized as at risk of, or having, CAR-T cell therapy-induced ARDS. In some embodiments, the patient has pneumonia, asthma, COPD and/or pulmonary fibrosis (all of which can put the patient as increased risk for ARDS and/or increased severity of ARDS).

As referred to herein, “prevention”, “prevent”, and/or “preventing”, which are used interchangeably herein, are intended to refer to all processes wherein there may be a slowing, interrupting, arresting, controlling, stopping, alleviating symptoms or complications or reversing of the progression of a respiratory disease, for example, caused by an injury, insult such as SARS-CoV-2 viral infection or disease such as COVID-19 disease, or therapy induced insult such as CAR-T therapy, whereby the respiratory disease does not progress to ARDS or does not progress to a more severe stage of ARDS, for example, as defined by the Berlin definition. As used herein, prevention is not intended to necessarily indicate a total elimination of all disorder symptoms.

As used interchangeably herein, “treatment” and/or “treating” and/or “treat” are intended to refer to all processes wherein there may be a slowing, interrupting, arresting, controlling, stopping, alleviating symptoms or complications or reversing of the progression of ARDS, but does not necessarily indicate a total elimination of all disorder symptoms.

As may be used herein, the terms “about” or “approximately”, when used in reference to a particular recited numerical value or range of values, means that the value may vary from the recited value by no more than 10% (e.g., +/−10%). For example, as used herein, the expression “about 100” includes 90 and 110 and all values in between (e.g., 91, 92, 93, 94, etc.).

### Anti-IL17 Antibodies

The present disclosure relates to monoclonal antibodies that specifically bind human IL-17 with high affinity. More specifically, the antibodies specifically bind and antagonize human IL-17A. According to specific embodiments, the an anti-IL17 antibody comprises two light chain variable regions (LCVR) each having the amino acid sequence SEQ ID NO: 2 and two heavy chain variable regions (HCVR) each having the amino acid sequence SEQ ID NO: 3. In more specific embodiments, the anti-IL17 antibody comprises two light chains (LC) each having the amino acid sequence SEQ ID NO: 4 and two heavy chains (HC) each having the amino acid sequence SEQ ID NO: 5, and wherein the HCs are cross-linked by disulfide bonds. In preferred embodiments of the present disclosure the anti-IL17 antibody is ixekizumab. Further characterization of antibodies for use in the present disclosure are provided in U.S. Pat. No. 7,838,638. Additional anti-IL17 antibodies are described in WO 2006/013107A1 and WO 2012/095662 A1.

### Pharmaceutical Formulation of Anti-IL17 Antibodies

Embodiments of the present disclosure also include the use of anti-IL17 antibodies incorporated in pharmaceutical formulations. A pharmaceutical formulation, as used herein, is a stable formulation comprising the anti-IL17 antibody of the present disclosure, preferably ixekizumab, wherein the degree of degradation, modification, aggregation, loss of biological activity and the like, of proteins/antibodies therein is acceptably controlled, and does not increase unacceptably with time. Stability of an antibody in solution depends on the chemical and physical stability of the antibody in the formulation in which the antibody is solubilized. Issues such as oxidation, deamidation, and hydrolysis are examples of chemical stability issues while aggregation, gel formation and liquid-liquid phase separation are examples of physical stability issues that an antibody can have in a formulation. Stability may be assessed by methods well-known in the art, including measurement of a sample's light scattering, apparent attenuation of light (absorbance, or optical density), size (e.g. by size exclusion chromatography (SEC)), in vitro or in vivo biological activity and/or properties measured by differential scanning calorimetry (DSC).

Pharmaceutical formulations for use in the present disclosure can be in the liquid dosage form of a solution, emulsion, or suspension and may be administered via parenteral administration including intravenous, intramuscular, subcutaneous, and intraperitoneal. Specific embodiments of anti-IL17 antibody pharmaceutical formulations include the pharmaceutical formulation of Table 1A and Table 1B.

TABLE 1A
Anti-IL-17 Antibody Pharmaceutical Formulation

| | Concentration |
|---|---|
| Component | (mg/mL) |
| Anti-IL-17 antibody* | 80 |
| Sodium Citrate Dihydrate | 5.106 |
| Citric Acid Anhydrous | 0.507 |
| Sodium Chloride | 11.69 |
| Polysorbate 80 | 0.30 |
| Water for Injection | q.s. to 1 mL |
| Hydrochloric Acid | pH adjustment |
| Sodium Hydroxide | pH adjustment |

TABLE 1B
Anti-IL-17 Antibody Pharmaceutical Formulation

| Component | Concentration | |
|---|---|---|
| Anti-IL-17 antibody* | 80 | mg/mL |
| Polysorbate 80 | 0.03% w/v | (0.3 mg/mL) |
| Sucrose | 234 mM | (8% w/v) |

| | |
|---|---|
| pH | 5.7 |

*The anti-IL17A antibody comprises a LCVR of SEQ ID NO: 2 and a HCVR of SEQ ID NO: 3.

Further characterization of anti-IL17 antibody pharmaceutical formulations for use in the present disclosure are provided in U.S. Pat. No. 9,376,491 and co-pending U.S. patent application Ser. No. 16/787,254. Preferred embodiments of the pharmaceutical formulations for use in the present disclosure include the anti-IL17 antibody being ixekizumab. Further embodiments include the commercially available pharmaceutical formulation Taltz®.

### Dose Regimen

The present disclosure also relates to dose regimens for the prevention and/or treatment of ARDS with anti-IL17 antibodies. As referred to herein, and as generally known in the art, the term “dose” refers to an amount (e.g., a concentration) of anti-IL17 antibody which is administered to a patient. A “dose regimen” or “dosage regimen” as generally known in the field and as may be referred to interchangeably herein includes a treatment schedule for administering a set (i.e., series or sequence) of doses to be administered to a patient over a period of time.

An exemplary embodiment of a dose regimen provided with the methods of preventing and/or treating herein, includes a single dose of about 160 mg of an anti-IL17 antibody or pharmaceutical formulation thereof. Further embodiments include an additional dose of 160 mg of anti-IL17 antibody or pharmaceutical formulation thereof each week thereafter (160Q1).

According to additional embodiments of dose regiments provided herewith, an initial dose of 160 mg of the anti-IL17 antibody or pharmaceutical formulation thereof is administered followed by 160 mg of the anti-IL17 antibody or pharmaceutical formulation thereof every two weeks thereafter (160Q2). In further embodiments of dose regiments provided herewith, an initial dose of 160 mg of the anti-IL17 antibody or pharmaceutical formulation thereof is administered followed by 160 mg of the anti-IL17 antibody or pharmaceutical formulation thereof every three weeks thereafter (160Q3). In yet further embodiments of dose regiments provided herewith, an initial dose of 160 mg of the anti-IL17 antibody or pharmaceutical formulation thereof is administered followed by 160 mg of the anti-IL17 antibody or pharmaceutical formulation thereof every four weeks thereafter (160Q4).

In yet even further exemplary embodiments, dose regiments provided herewith include an initial dose of 160 mg of the anti-IL17 antibody or pharmaceutical formulation thereof followed by 80 mg of the anti-IL17 antibody or pharmaceutical formulation thereof administered every week thereafter. In other embodiments, dose regiments provided herewith include an initial dose of 160 mg of the anti-IL17 antibody or pharmaceutical formulation thereof followed by 80 mg of the anti-IL17 antibody or pharmaceutical formulation thereof administered every two weeks thereafter. Further, exemplary embodiments of dose regiments provided herewith include an initial dose of 160 mg of the anti-IL17 antibody or pharmaceutical formulation thereof followed by 80 mg of the anti-IL17 antibody or pharmaceutical formulation thereof administered every three weeks thereafter. Even further, an exemplary embodiment of a dose regimen provided herewith includes a dose regiment comprising an initial dose of 160 mg of the anti-IL17 antibody or pharmaceutical formulation thereof followed by 80 mg of the anti-IL17 antibody or pharmaceutical formulation thereof administered every four weeks thereafter.

Additionally, a dose regimen provided with the methods of preventing and/or treating herein, includes a single dose of about 80 mg of an anti-IL17 antibody or pharmaceutical formulation thereof. Further embodiments include an additional dose of 80 mg of anti-IL17 antibody or pharmaceutical formulation thereof each week thereafter (80Q1).

According to additional embodiments of dose regiments provided herewith, an initial dose of 80 mg of the anti-IL17 antibody or pharmaceutical formulation thereof is administered followed by 80 mg of the anti-IL17 antibody or pharmaceutical formulation thereof every two weeks thereafter (80Q2). In further embodiments of dose regiments provided herewith, an initial dose of 80 mg of the anti-IL17 antibody or pharmaceutical formulation thereof is administered followed by 80 mg of the anti-IL17 antibody or pharmaceutical formulation thereof every three weeks thereafter (80Q3). In yet further embodiments of dose regiments provided herewith, an initial dose of 80 mg of the anti-IL17 antibody or pharmaceutical formulation thereof is administered followed by 80 mg of the anti-IL17 antibody or pharmaceutical formulation thereof every four weeks thereafter (80Q4).

### EXAMPLES

### Example 1. IL-17 Pathway Biomarker Study

Diffuse alveolar damage, due to infection with the H1N1 virus, has been associated with a cytotoxic inflammatory phenotype. IL-17 pathway is a possible mediator of such damage. Further, the cytokine storm may include potential IL-17 pathway mediators possibly including IL-22, IL-23, IL-19, CCL20 (also known as CCR6 ligands) and CCL2, CCL4, CCL5, CCL17 and CCL22 (also known as CCR4 ligands). Furthermore, ixekizumab plays a role in IL-4 regulation, a cell survival regulator which may play a role in ARDS development or progression. As such, a study of biomarker impact of patients with respiratory disease, resulting from COVID-19, may be undertaken according to the following. Briefly, blood samples and/or bronchioalveolar lavage samples from patients with COVID-19 may be obtained at different time points including: prior to infection, during infection, prior to onset of ARDS, during mild ARDS, prior to ICU admittance, following ICU admittance, post ICU release and/or following subsiding of ARDS. For example, a study may include collection of blood samples and/or bronchioalveolar lavage samples from patients with COVID-19 prior to ICU admittance, following ICU admittance and post ICU release. Samples may be tested for biomarkers implicated in IL-17 pathway, including IL-19, IL-22, IL-23, IL-4, CCR4 and/or CCR6. Results may demonstrate IL-17 impact in progression of respiratory disease resulting in ARDS or resulting in increasing severity of ARDS.

Following methods substantially as described above, the fold change increase in IL-17A, IL-17C and IL-6 is assessed in ARDS patients (both COVID-19 positive (N=58) and COVID-19 negative (N=22)) and compared to control healthy (both non-ARDS and non-COVID-19) patients (N=20). Serum samples are analyzed in duplicate using multiplex immunoassay. Compared to healthy patients: (i) COVID-19 positive ARDS patients demonstrated a 1.75 fold increase in IL-17A, a 1.91 fold increase in IL-17C, and a 14.49 fold increase in IL-6; and (ii) COVID-19 negative ARDS patients demonstrated a 2.61 fold increase in IL-17A, a 2.08 fold increase in IL-17C, and a 16.17 fold increase in IL-6.

### Example 2. Clinical Study of Ixekizumab in COVID-19 Patients

A clinical study comparing prevention and/or treatment of ARDS in patients with COVID-19 with ixekizumab (an anti-IL-17 antibody having a LC with the amino acid sequence of SEQ ID NO. 4 and a HC with the amino acid sequence of SEQ ID NO. 5, and further described in U.S. Pat. Nos. 7,838,638 and 9,376,491), may be undertaken as described herein. Briefly, patients positive for COVID-19 may be administered one of 160 mg or 80 mg of ixekizumab on day 0. Thereafter, patients may be administered one of 160 mg or 80 mg of ixekizumab the following week (e.g., 160Q1 or 80Q1), two weeks (e.g., 160Q2 or 80Q2), three weeks (e.g., 160Q3 or 80Q3) or four weeks (e.g., 160Q4 or 80Q4). Patients may be assessed for respiratory disease presence, ARDS presence, or respiratory disease or ARDS progression during and following treatment. Assessment may include chest imagining and PaO2/FiO2 ratio assessment.

According to a clinical study, assessment of prevention and/or treatment of ARDS in patients with COVID-19 with ixekizumab (an anti-IL-17 antibody having a LC with the amino acid sequence of SEQ ID NO. 4 and a HC with the amino acid sequence of SEQ ID NO. 5, and further described in U.S. Pat. Nos. 7,838,638 and 9,376,491) may be undertaken as follows. Patients positive for COVID-19 may be administered 160 mg of ixekizumab by subcutaneous injection on day 0. Two weeks thereafter (e.g., day 14), patients may be subcutaneously administered 80 mg of ixekizumab (unless prior to day 14 patient treatment is discontinued). Patients completing dosing on day 0 and day 14, or day 0 and discontinued, may be assessed for one or more of clinical recovery time, oxygen free recovery rate (e.g., discharged from hospitalization and not receiving supplemental oxygen), disease severity ordinal scale, mortality, respiratory disease presence, ARDS presence, or respiratory disease or ARDS progression during and following treatment. Assessment may include chest imagining and PaO2/FiO2 ratio assessment.

### Exemplified Embodiments

The below provides additional embodiments set forth throughout the disclosure.
1. A method of preventing acute respiratory distress syndrome (ARDS) in a patient comprising administering to said patient a therapeutically effective amount of an anti-IL17 antibody, or a pharmaceutical formulation thereof.
2. The embodiment of 1, wherein the patient is at risk of developing ARDS.
3. The embodiment of any of 1-2, wherein the patient has a respiratory insult.
4. The embodiment of 3, wherein the respiratory insult is a respiratory disease.
5. The embodiment of 3, wherein the respiratory insult is a respiratory injury.
6. A method of treating ARDS in a patient comprising administering to said patient a therapeutically effective amount of an anti-IL17 antibody, or a pharmaceutical formulation thereof.
7. The embodiment of 6, wherein the patient is diagnosed as having mild ARDS.
8. The embodiment of 6, wherein the patient is diagnosed as having moderate ARDS.
9. The embodiment of 6, wherein the patient is diagnosed as having severe ARDS.
10. The embodiment of any of 7-9, wherein the patient is diagnosed as having one of mild, moderate or severe ARDS according to the Berlin definition.
11. The embodiment of any of 1-10, wherein the patient has a viral infection.
12. The embodiment of 11, wherein the viral infection is a coronavirus infection.
13. The embodiment of 12, wherein the coronavirus is SARS-CoV-2.
14. The embodiment of any of 1-3, wherein the patient has pneumonia, asthma, COPD, interstitial lung disease and/or pulmonary fibrosis.
15. The embodiment of any of 1-4, wherein said anti-IL17 antibody specifically binds human IL17A.
16. The embodiment of any of 1-15, wherein the anti-IL17 antibody comprises a light chain variable region (LCVR) and a heavy chain variable region (HCVR), wherein said LCVR is the amino acid sequence of SEQ ID NO: 2 and said HCVR is the amino acid sequence of SEQ ID NO: 3.
17. The embodiment of 15, wherein the anti-IL17 antibody comprises a light chain (LC) and a heavy chain (HC), wherein said LC is the amino acid sequence of SEQ ID NO: 4 and said HC is the amino acid sequence of SEQ ID NO: 5.
18. The embodiment of 17, wherein the anti-IL17 antibody is ixekizumab.
19. The embodiment of any of 1-18, wherein administering comprises administering a pharmaceutical formulation of the anti-IL17 antibody, said pharmaceutical formulation comprising the anti-IL17 antibody at a concentration of about 80 mg/mL.
20. The embodiment of any of 1-18, wherein administering comprises administering a pharmaceutical formulation of the anti-IL17 antibody, said pharmaceutical formulation comprising the anti-IL17 antibody at a concentration of about 160 mg/ml.
21. The embodiment of any of 19-20, wherein the pharmaceutical formulation further comprises a citrate buffer at a concentration of about 20 mM, sodium chloride at a concentration of about 200 mM, polysorbate-80 at a concentration of about 0.03% (w/v) and pH at about 5.7.
22. The embodiment of any of 19-20, wherein the pharmaceutical formulation further comprises sucrose at a concentration of about 234 mM, polysorbate-80 at a concentration of about 0.03% (w/v) and pH at about 5.7, wherein the pharmaceutical formulation is substantially free of an ionic tonicity excipient and substantially free of L-amino acid excipients.
23. The embodiment of any of 1-22, wherein administering comprises administering a dose of about 160 mg of the anti-IL17 antibody.
24. The embodiment of any of 1-22, wherein administering comprises administering a dose of about 80 mg of the anti-IL17 antibody.
25. The embodiment of any of 23-24, wherein administering further comprises administering about 160 mg of the anti-IL17 antibody at each of weeks 2, 3 and 4 following the initial dose (e.g., 160Q1).
26. The embodiment of any of 23-24, wherein administering further comprises administering about 80 mg of the anti-IL17 antibody at each of weeks 1, 2, 3 and 4 following the initial dose (e.g., 80Q1).
27. The embodiment of any of 23-24, wherein administering further comprises administering about 160 mg of the anti-IL17 antibody or pharmaceutical formulation thereof at each of Weeks 2 and 4 following the initial dose (e.g., 160Q2).
28. The embodiment of any of 23-24, wherein administering further comprises administering about 80 mg of the anti-IL17 antibody or pharmaceutical formulation thereof at each of Weeks 2 and 4 following the initial dose (e.g., 80Q2).
29. The embodiment of any of 23-24, wherein administering further comprises administering about 160 mg of the anti-IL17 antibody or pharmaceutical formulation thereof at week 4 following the initial dose (e.g., 160Q4).
30. The embodiment of any of 23-24, wherein administering further comprises administering about 80 mg of the anti-IL17 antibody or pharmaceutical formulation thereof at week 4 following the initial dose (e.g., 80Q4).
31. The embodiment of any of 1-29, wherein said step of administering comprises subcutaneous administration.
32. An anti-IL17 antibody, or a pharmaceutical formulation thereof, for use in the manufacture of a medicament for the prevention of ARDS.
33. An anti-IL17 antibody, or a pharmaceutical formulation thereof, for use in the manufacture of a medicament for the treatment of ARDS.
34. An anti-IL17 antibody, or a pharmaceutical formulation thereof, for use in the prevention of ARDS, wherein a therapeutically effective amount of the anti-IL17 antibody or a pharmaceutical formulation thereof is administered to a patient at risk of ARDS.
35. The anti-IL17 antibody, or pharmaceutical formulation thereof, of the embodiment of 34, wherein the patient has a respiratory insult.
36. The anti-IL17 antibody, or pharmaceutical formulation thereof, of the embodiment of 35, wherein the respiratory insult is a respiratory disease.
37. The anti-IL17 antibody, or pharmaceutical formulation thereof, of the embodiment of 35, wherein the respiratory insult is a respiratory injury.
38. An anti-IL17 antibody, or a pharmaceutical formulation thereof, for use in the treatment of ARDS, wherein a therapeutically effective amount of the anti-IL17 antibody, or pharmaceutical formulation thereof, is administered to a patient in need of treatment for ARDS.
39. The anti-IL17 antibody, or pharmaceutical formulation thereof, of the embodiment of 38, wherein the patient is diagnosed as having mild ARDS.
40. The anti-IL17 antibody, or pharmaceutical formulation thereof, of the embodiment of 38, wherein the patient is diagnosed as having moderate ARDS.
41. The anti-IL17 antibody, or pharmaceutical formulation thereof, of the embodiment of 38, wherein the patient is diagnosed as having severe ARDS.
42. The anti-IL17 antibody, or pharmaceutical formulation thereof, of any of the embodiment of any of 38-41, wherein the patient is diagnosed as having one of mild, moderate or severe ARDS according to the Berlin definition.
43. The anti-IL17 antibody, or pharmaceutical formulation thereof, of the embodiment of any of 34-42, wherein the patient has a viral infection.
44. The anti-IL17 antibody, or pharmaceutical formulation thereof, of the embodiment of 43, wherein the viral infection is a coronavirus infection.
45. The anti-IL17 antibody, or pharmaceutical formulation thereof, of the embodiment of 44, wherein the coronavirus is SARS-CoV-2.
46. The anti-IL17 antibody, or pharmaceutical formulation thereof, of the embodiment of any of 34-45, wherein the patient has pneumonia, asthma, COPD, interstitial lung disease and/or pulmonary fibrosis.
47. The anti-IL17 antibody of the embodiment of any of 32-46, wherein said anti-IL17 antibody specifically binds human IL17A.
48. The anti-IL17 antibody of the embodiment of any of 32-47, wherein the anti-IL17 antibody comprises a light chain variable region (LCVR) and a heavy chain variable region (HCVR), wherein said LCVR is the amino acid sequence of SEQ ID NO: 2 and said HCVR is the amino acid sequence of SEQ ID NO: 3.
49. The anti-IL17 antibody of the embodiment of 48, wherein the anti-IL17 antibody comprises a light chain (LC) and a heavy chain (HC), wherein said LC is the amino acid sequence of SEQ ID NO: 4 and said HC is the amino acid sequence of SEQ ID NO: 5.
50. The anti-IL17 antibody of the embodiment of 49, wherein the anti-IL17 antibody is ixekizumab.
51. The anti-IL17 antibody, or pharmaceutical formulation thereof, of the embodiment of any of 32-46 comprising a pharmaceutical formulation of the anti-IL17 antibody, said pharmaceutical formulation comprising the anti-IL17 antibody at a concentration of about 80 mg/mL, citrate buffer at a concentration of about 20 mM, sodium chloride at a concentration of about 200 mM, polysorbate-80 at a concentration in the range of about 0.03% (w/v), and pH at about 5.7.
52. The anti-IL17 antibody, or pharmaceutical formulation thereof, of the embodiment of any of 32-46 comprising a pharmaceutical formulation of the anti-IL17 antibody, said pharmaceutical formulation comprising the anti-IL17 antibody at a concentration of about 80 mg/mL, sucrose at a concentration of about 234 mM, polysorbate-80 at a concentration of about 0.03% (w/v) and pH at about 5.7, wherein the pharmaceutical formulation is substantially free of an ionic tonicity excipient and substantially free of L-amino acid excipients.

Sequence Listing
(human IL-17)
SEQ ID NO: 1
MTPGKTSLVSLLLLLSLEAIVKAGITIPRNPGCPNSEDKNFPRTVMVNLN
IHNRNTNTNPKRSSDYYNRSTSPWNLHRNEDPERYPSVIWEAKCRHLGCI
NADGNVDYHMNSVPIQQEILVLRREPPHCPNSFRLEKILVSVGCTCVTPI
VHHVA
(LCVR)
SEQ ID NO: 2
DIVMTQTPLSLSVTPGQPASISCRSSRSLVHSRGNTYLHWYLQKPGQSPQ
LLIYKVSNRFIGVPDRFSGSGSGTDFTLKISRVEAEDVGVYYCSQSTHLP
FTFGQGTKLEIK
(HCVR)
SEQ ID NO: 3
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTLVTVSS
(Light chain)
SEQ ID NO: 4
DIVMTQTPLSLSVTPGQPASISCRSSRSLVHSRGNTYLHWYLQKPGQSPQ
LLIYKVSNRFIGVPDRFSGSGSGTDFTLKISRVEAEDVGVYYCSQSTHLP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(Heavy chain)
SEQ ID NO: 5
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG

1. A method of preventing acute respiratory distress syndrome (ARDS) in a patient at risk of developing ARDS comprising administering to said patient a therapeutically effective amount of an anti-IL17 antibody, or a pharmaceutical formulation thereof,
wherein the anti-IL17 antibody comprises a light chain variable region (LCVR) and a heavy chain variable region (HCVR), wherein said LCVR is the amino acid sequence of SEQ ID NO: 2 and said HCVR is the amino acid sequence of SEQ ID NO: 3.

2. (canceled)

3. The method of claim 3, wherein the patient has a respiratory insult.

4. The method of claim 3, wherein the respiratory insult is a respiratory disease or a respiratory injury.

5-9. (canceled)

10. The method of claim 1, wherein the patient has a viral infection.

11. The method of claim 10, wherein the viral infection is a coronavirus infection.

12. The method of claim 11, wherein the coronavirus is SARS-CoV-2.

13. The method of claim 1, wherein the patient has pneumonia, asthma, COPD, interstitial lung disease and/or pulmonary fibrosis.

14-15. (canceled)

16. The method of claim 1, wherein the anti-IL17 antibody comprises a light chain (LC) and a heavy chain (HC), wherein said LC is the amino acid sequence of SEQ ID NO: 4 and said HC is the amino acid sequence of SEQ ID NO: 5.

17. The method of claim 16, wherein the anti-IL17 antibody is ixekizumab.

18-19. (canceled)

20. The method of claim 1, wherein administering comprises:
(i) administering a dose of about 160 mg of the anti-IL17 antibody;
(ii) administering a dose of about 80 mg of the anti-IL17 antibody;
(iii) administering about 160 mg of the anti-IL17 antibody at each of weeks 2, 3 and 4 following the initial dose (e.g., 160Q1);
(iv) administering about 80 mg of the anti-IL17 antibody at each of weeks 1, 2, 3 and 4 following the initial dose (e.g., 80Q1);
(v) administering about 160 mg of the anti-IL17 antibody or pharmaceutical formulation thereof at each of Weeks 2 and 4 following the initial dose (e.g., 160Q;
(vi) administering about 80 mg of the anti-IL17 antibody or pharmaceutical formulation thereof at each of Weeks 2 and 4 following the initial dose (e.g., 80Q2);
(vii) administering about 160 mg of the anti-IL17 antibody or pharmaceutical formulation thereof at week 4 following the initial dose (e.g., 160Q4); or
(iix) administering about 80 mg of the anti-IL17 antibody or pharmaceutical formulation thereof at week 4 following the initial dose (e.g., 80Q4).

21. The method of claim 1, wherein said step of administering comprises subcutaneous administration.

22. A method of treating acute respiratory distress syndrome (ARDS) in a patient comprising administering to said patient a therapeutically effective amount of an anti-IL17 antibody, or a pharmaceutical formulation thereof, wherein the anti-IL17 antibody comprises a light chain variable region (LCVR) and a heavy chain variable region (HCVR), wherein said LCVR is the amino acid sequence of SEQ ID NO: 2 and said HCVR is the amino acid sequence of SEQ ID NO: 3.

23. The method of claim 22, wherein the patient is diagnosed as having one of mild ARDS, moderate ARDS or severe ARDS.

24. The method of claim 23, wherein the patient is diagnosed as having one of mild, moderate or severe ARDS according to the Berlin definition.

25. The method of claim 22, wherein the patient has a viral infection.

26. The method of claim 25, wherein the viral infection is a coronavirus infection.

27. The method of claim 26, wherein the coronavirus is SARS-CoV-2.

28. The method of claim 22, wherein the anti-IL17 antibody comprises a light chain (LC) and a heavy chain (HC), wherein said LC is the amino acid sequence of SEQ ID NO: 4 and said HC is the amino acid sequence of SEQ ID NO: 5.

29. The method of claim 28, wherein the anti-IL17 antibody is ixekizumab.

30. The method of claim 22, wherein said step of administering comprises subcutaneous administration.

31. The method of claim 30, wherein administering comprises:
(i) administering a dose of about 160 mg of the anti-IL17 antibody;
(ii) administering a dose of about 80 mg of the anti-IL17 antibody;
(iii) administering about 160 mg of the anti-IL17 antibody at each of weeks 2, 3 and 4 following the initial dose (e.g., 160Q1);
(iv) administering about 80 mg of the anti-IL17 antibody at each of weeks 1, 2, 3 and 4 following the initial dose (e.g., 80Q1);
(v) administering about 160 mg of the anti-IL17 antibody or pharmaceutical formulation thereof at each of Weeks 2 and 4 following the initial dose (e.g., 160Q1);
(vi) administering about 80 mg of the anti-IL17 antibody or pharmaceutical formulation thereof at each of Weeks 2 and 4 following the initial dose (e.g., 80Q2);
(vii) administering about 160 mg of the anti-IL17 antibody or pharmaceutical formulation thereof at week 4 following the initial dose (e.g., 160Q4); or
(iix) administering about 80 mg of the anti-IL17 antibody or pharmaceutical formulation thereof at week 4 following the initial dose (e.g., 80Q4).


SEQUENCE LISTING (5 sequences)

SEQ ID NO: 1 - protein, 155 aa, synthetic construct
  REGION 1..155: note = hIL-17
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 2 - protein, 112 aa, synthetic construct
  REGION 1..112: note = LCVR
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIK

SEQ ID NO: 3 - protein, 119 aa, synthetic construct
  REGION 1..119: note = HCVR
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSS

SEQ ID NO: 4 - protein, 219 aa, synthetic construct
  REGION 1..219: note = Light chain
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 445 aa, synthetic construct
  REGION 1..445: note = Heavy chain
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG